(19) United States
(12) Patent Application Publication
(10) Pub. No.: US 2008/0269580 A1
(43) Pub. Date: Oct. 30, 2008

(54) System for Non-Invasive Measurement of Bloold Glucose Concentration

(75) Inventors: Marcello Leonardo Mario Balistreri, Eindhoven (NL); Maarten Marinus Johannes Wilhelmus Van Herpen, Eindhoven (NL); Antonius Theodorus Martinus Van Gogh, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS, N.V., EINDHOVEN (NL)

(21) Appl. No.: 12/158,469

(22) PCT Filed: Dec. 12, 2006
(86) PCT No.: PCT/IB2006/054773

§ 371 Date: Jun. 20, 2008

(30) Foreign Application Priority Data
Dec. 22, 2005 (EP) 05301095.5

(51) Int. Cl. A61B 5/01; A61B 5/1468; A61B 8/06

(52) U.S. Cl. 600/328; 600/549; 600/438

(57) ABSTRACT

A system and method for non-invasive measurement of glucose concentration in a live subject including a thermal emission spectroscopy (TES) device 10, an optical coherence tomography (OCT) device 20 or near infrared diffuse reflectance (NIDR) device. The TES 10 generates a signal indicative of the absorbtion of glucose, from which the blood glucose concentration is determined and the OCT device 20 generates a signal indicative of the scattering coefficient of a portion of the live subject, from which the blood glucose concentration is determined. The signals generated by the TES and OCT devices along with signals generated by sensors for measuring the body heat and surface temperature of the subject are used in the metabolic heat conformation (MHC) method of determining blood glucose concentration. The system may include a photoacoustic sensor for generating a signal indicative of thermo-elastic skin properties from which the blood glucose concentration is also determined.

The present invention relates to a system for the non-invasive measurement of blood glucose concentration in a live subject.

The determination of blood glucose concentration is frequently done invasively by taking a blood sample and transferring the sample to a laboratory or a hand-held device where it is analysed. Measuring blood glucose concentration in-vivo is complicated by the interference of several physiological and other variables which can completely overwhelm the blood glucose signal. It is very difficult to eliminate these interferences as they may contribute non-linearly to the measured signal, they may vary with the spatial location from the subject, they may vary over time or may vary from person to person.

One non-invasive method of determining blood glucose concentration uses the known Metabolic Heat Conformation (MHC) method as described by Cho et al. (“Non-invasive Measurement of Glucose by Metabolic Heat Conformation Method”, Clinical Chemistry, 50(10), pp 1894-1898, (2004)). This method relies on the measurement of the oxidative metabolism of glucose, from which the blood glucose concentration can be inferred. Body heat generated by glucose oxidation is based on the subtle balance of capillary glucose and oxygen supply to the cells of a tissue. The MHC method exploits this relationship to estimate blood glucose by measuring the body heat and the oxygen supply. The relationship can be represented in an equation as:

[Glucose concentration]=Function[Heat generated, Blood flow rate, Hb, HbO₂]

where Hb and HbO₂ represent the haemoglobin and oxygenated haemoglobin concentrations, respectively.

The heat generated (i.e. body heat) is measured with a thermometer and the Hb and HbO₂ concentrations are typically determined from the spectral reflectivity of the skin. Using the known MHC method, the blood flow rate is estimated from the thermal conductivity of the skin, and this thermal conductivity is detected by measuring the heat transferred through the skin from the tissue sample, such as a fingertip, to two thermistors.

The accuracy of the measurement of glucose concentration using the MHC method thus depends on various measurements each with associated inaccuracies including the thermal conductivity of the skin, which depends on the water content of the tissue sample. Unless the water content is determined first, the inaccuracy associated with the calculated blood flow rate in particular can become quite large.

It is an object of the present invention to provide a system for the non-invasive measurement of blood glucose concentration in a live subject which provides improved accuracy over the known MHC method.

In accordance with the present invention there is provided a system for the non-invasive measurement of blood glucose concentration in a live subject comprising:
a. means for determining the body heat of the subject,
b. means for determining the concentration of haemoglobin and oxygenated haemoglobin in the blood of said live subject, and
c. means for determining blood flow velocity in respect of said live subject and means for determining blood glucose concentration in said live subject as a function of said body heat, said haemoglobin and oxygenated haemoglobin concentrations and said blood flow velocity; and
a plurality of spectroscopic devices each generating a signal indicative of blood glucose concentration, means for determining the blood glucose concentration from the signal indicative of blood glucose concentration and wherein at least one of the spectroscopic devices generates a signal additionally indicative of one or more of:
d. concentration of haemoglobin and oxygenated haemoglobin in the blood of the live subject;
e. the body heat of the live subject;
f. ambient temperature;
g. blood flow velocity in respect of said live subject,
   wherein the signals indicative of one or more of d. to g. are transmitted to at least one of means a. to c. and used to determine the blood glucose concentration.

The system of the present invention extracts information spectroscopically in addition to implementing the MHC method to enable values of blood glucose concentration to be determined. The blood glucose concentration values determined thus have less interference in common and can be used to compensate weaknesses or interferences of one technique using the information from another. A more accurate determination of blood glucose concentration can thus be achieved.

Preferably one of the spectroscopic devices comprises

x) a detector for detecting the thermal emission spectrum emitted by said live subject and generating a signal indicative of the absorption of glucose.

Thermal Emission Spectroscopy (TES) is one method of non-invasively determining glucose concentration as disclosed in e.g. U.S. Pat. No. 5,666,956. With this method, the thermal or blackbody radiation of the human body is measured in the infrared part of the electromagnetic spectrum; the resulting intensity and spectral measurements are found to be characteristic of the temperature and state of the radiating object.

Preferably one of the spectroscopic devices comprises

y) an irradiator for irradiating a portion of the live subject with a measuring beam in and a detector for collecting measuring beam radiation scattered by said live subject and generating a signal indicative of the scattering coefficient of the portion of the subject.

Preferably the measuring beam is in the near infrared spectrum and more preferably has multiple wavelengths.

Examples of such devices are an optical coherence tomography (OCT) device, an optical Doppler tomography device or near infrared diffuse reflectance (NIDR) devices. The term “near infrared” is used to refer to light of wavelengths between 0.70 and 2.5 μm.

More preferably the spectroscopic device comprises interference filtering means for spatially separating said thermal emission spectrum to create a plurality of spectral patterns and measuring in respect of each of a plurality of said spectral patterns a spectral intensity at a first, reference set of wavelengths, and a second set of wavelengths dependent on glucose or other analyte, and the concentration of glucose or other analyte is determined therefrom.

Measuring the reference and glucose signals at a plurality of wavelengths and in other parts of the spectrum means that more information is obtained, resulting in a better accuracy of the glucose concentration. Measuring parts of the spectrum containing information of other analytes allows for the correction for the interference from other analytes, thereby further increasing the accuracy of the glucose concentration measurement.

Preferably the signal generated by the detector of x) is also indicative of the concentration of haemoglobin and oxygenated haemoglobin in the blood of the live subject.

The signal indicative of the concentration haemoglobin and oxygenated haemoglobin can be used as an input signal for the means for determining the concentration of the haemoglobin and oxygenated haemoglobin.

Preferably the signal generated by the detector of x) is also indicative of the body heat of the live subject. The signal indicative of the body heat can be used as an input signal for the means for determining the body heat of the subject.

Preferably the signal generated by the detector of x) is also indicative of the ambient temperature.

The signal indicative of the ambient temperature can be used as an input signal for the means for determining the body heat of the subject.

Preferably the signal generated by the detector of y) is indicative of the blood flow velocity in respect of said live subject. The signal indicative of the blood flow velocity of the live subject can be used as a relatively high accuracy input signal for the means for determining blood flow velocity in respect of the live subject.

The present invention also relates to a method of determining blood glucose concentration in a live subject non-invasively comprising the steps of:
m. determining the body heat of the subject,
n. determining the concentration of haemoglobin and oxygenated haemoglobin in the blood of said live subject, and
o. determining blood flow velocity in respect of said live subject and means for determining blood glucose concentration in said live subject as a function of said body heat, said haemoglobin and oxygenated haemoglobin concentrations and said blood flow velocity; and
generating a signal indicative of blood glucose concentration from a plurality of spectroscopic devices and determining the blood glucose concentration therefrom, at least one signal being additionally indicative of one or more of:
p. concentration of haemoglobin and oxygenated haemoglobin in the blood of the live subject;
q. the body heat of the live subject;
r. ambient temperature;
s. blood flow velocity in respect of said live subject,
   and using the signal(s) indicative of one or more of p. to s. in at least one of steps m to o.

These and other aspects of the present invention will be apparent from and elucidated with reference to the embodiments described herein.

Embodiments of the present invention will now be described by way of examples only and with reference to the accompanying schematic drawings in which:

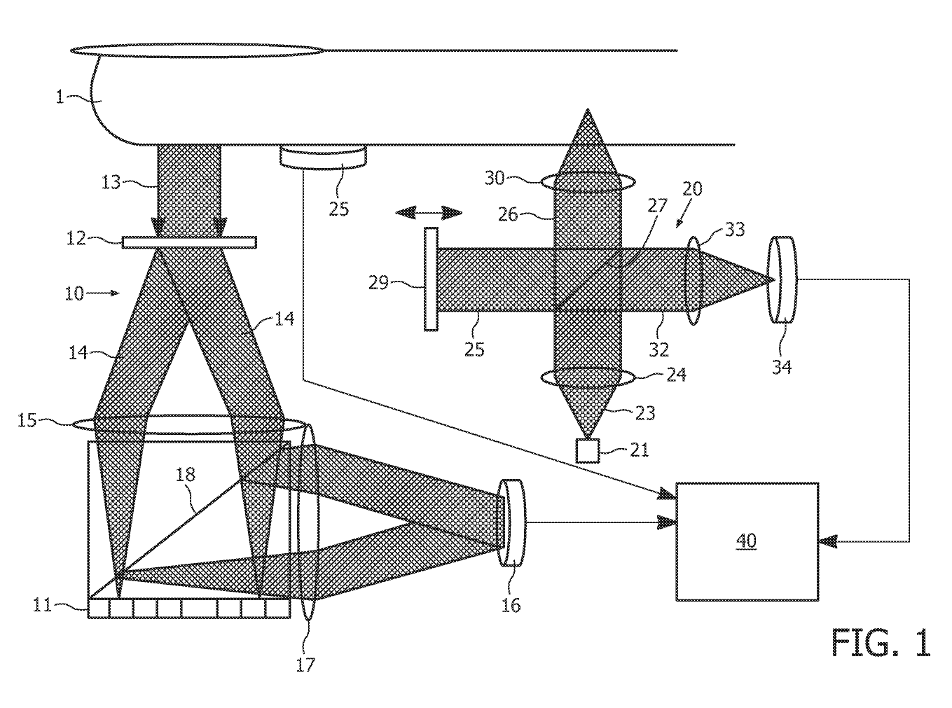

FIG. 1 shows a first embodiment of the system of the invention;

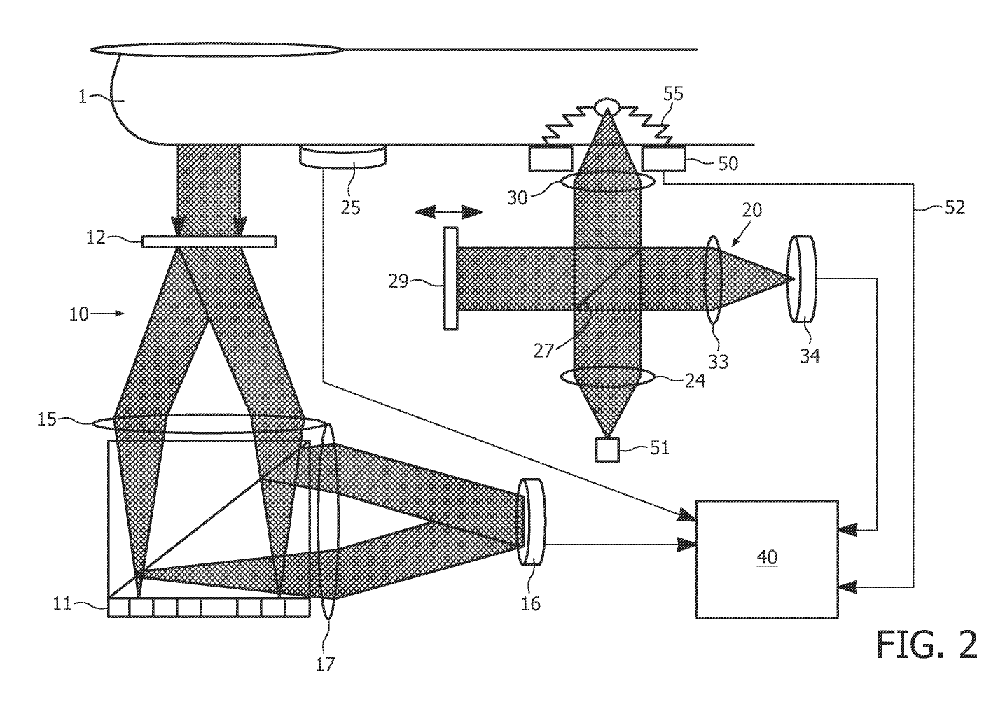

FIG. 2 shows a second embodiment of the system of the invention.

Referring to FIG. 1 the system is shown applied to a finger 1 of a live subject. The system includes a simplified thermal emission spectroscopy (TES) based device 10 in which a spatial light modulator (SLM) 11 such as a liquid crystal panel, a digital mirror display or a liquid crystal on a silicon display (LCOS display), is used in conjunction with a diffraction grating 12.

The blackbody radiation 13 (i.e. thermal emission spectrum) emanating from the finger 1 is spatially organised according to the constituent wavelengths 14, by the diffraction grating 12. The grating 12 splits the spatially mixed spectrum of wavelengths 13 and spatially re-arranges the spectrum in order of the wavelengths constituting the spectrum. This “organised spectrum” 14 is then focussed onto the SLM 11 by a first lens system 15.

The various parts of the organised spectrum 14 can be analysed by assigning grey levels to specific pixels of the SLM 11. For example, making a collection of pixels black at a given location on the SLM 11, will prevent those wavelengths of the “organised spectrum” 14, incident upon the blackened pixels, from being reflected by the SLM 11. Conversely, making a collection of pixels white will allow those wavelengths incident thereon to be reflected by the SLM 11. The wavelengths reflected from the SLM 11 are focussed onto a detector 16 via polarizing beam splitter 18, using a second lens system 17. In this manner, parts of the spectrum 14 can be reflected and others blocked. Thus, by switching certain wavelengths on and off, glucose signature spectral bands and spectral bands for reference measurements can be measured sequentially. Alternatively, by using more than one detector or a detector array, many signals can be measured simultaneously.

The SLM may also be used in a transmission setup with lens system 17 and detector 16 positioned in line with lens system 15.

The present embodiment is also amenable to multivariate calibration methods such as partial least squares regression. Such methods take into account the variation in the entire thermal emission spectrum 13 signal to allow the maximum amount of information to be extracted from the spectrum.

The multivariate calibration procedure produces a regression vector r=[r(λ₁), . . . , r(λₙ)], where r(λₙ) is a weighting function as applied to wavelength λₙ of the thermal emission spectrum 13, for an analyte of interest, e.g. glucose. (Wavelengths λ₁ to λₙ correspond to those wavelengths present in the emission spectrum). Subsequently taking the inner product of the regression vector with the measured thermal emission spectrum s=[s(λ₁), . . . , s(λₙ)] gives the concentration of the analyte of interest, in this case glucose.

The multivariate calibration method proceeds by displaying the weighting factors r(λ₁) to r(λₙ) on the pixels of the SLM 11 and subsequently focussing those wavelengths transmitted through the SLM 11 onto the detector 16 using the second lens system 17. Similarly, other desired signal patterns can also be extracted by displaying other regression vectors on the SLM 11. In this way, the glucose absorption and reference measurements can be made at more than one wavelength to improve the accuracy in the measurements. The SLM 11 acts as a so-called Multivariate Optical Element (MOE). However, when only one signal component is required, the MOE does not need to be adjusted and so the cheaper alternative of an interference filter can be used as a MOE.

Detector 16 generates a signal indicative of the absorption of glucose and the signal is transmitted to processor 40 which determines the blood glucose concentration therefrom. As well as generating a signal indicative of the absorption of glucose the TES device can also be used to generate a signal indicative of other blood constituents, such as haemoglobin and oxygenated-haemoglobin.

In addition, as an alternative to a thermometer, the generated signal from the TES detector 16 can also be used to determine the heat generated in the skin, using the temperature dependence of the blackbody curve given by the Planck energy distribution formula:

The detector 16 can also be used to generate a signal indicative of the ambient temperature by using the SLM 11 to keep radiation from the finger 1 away from the

$$\begin{matrix} \\ {P}_{λ} = \frac{2 π {hc}^{2}}{{λ}^{5} ( {e}^{( hc / λ kt )} - 1 )} \\ \end{matrix}$$

$$\begin{matrix}{P}_{λ} = Power per {m}^{2} area per m wavelength \\ h = Planck ' s constant ( 6.626 \times {10}^{- 34} Js ) \\ c = Speed of Light ( 3 \times {10}^{8} m \text{/} s ) \\ λ = Wavelength ( m ) \\ k = Boltzmann Constant ( 1.38 \times {10}^{- 23} J \text{/} K ) \\ t = Temperature ( K )\end{matrix}$$

detector.

The system also comprises an optical coherence tomography (OCT) device 20. The device 20 includes super luminescent diode (SLD) 21 as a broadband light source, i.e. a source that can emit light over a broad range of frequencies. A laser with extremely short pulses (femtosecond laser) is also suitable. The light 23 emitted by the SLD passes through collimating lens 24 and is split into two arms, reference arm 25 and sample arm 26, by 50/50 beam splitter 27. Reference arm 25 is directed towards and reflected from mirror 29. The mirror 29 can be scanned to change the pathlength of reference arm 25 over time. Sample arm 26 is directed towards finger 1, the sample in this case, and is focused by lens 30 onto finger 1. Backscattered light from the finger and reflected reference light from the reference arm are combined in beam 32 and interfere. The presence of glucose decreases the scattering coefficient of the tissue of finger 1. Detector 34 generates a signal indicative of the scattering coefficient, which is transmitted to processor 40, which determines the blood glucose concentration therefrom. Scanning mirror 29 allows a reflectivity profile of the sample to be obtained. U.S. Pat. No. 6,725,073 discloses methods for measuring analyte concentration within a tissue using optical coherence tomography.

The reflection of waves off a moving object is known to cause a frequency shift (the typical example being the change in the tone of a police car siren as the car approaches and then moves away), from which the speed of the moving object can be determined. Thus, due to the interaction of the radiation with the moving red blood cells within the radiated tissue sample and maybe the pulsating surface of the tissue sample, some regions of the radiation will suffer a frequency shift causing the intensity of the backscattered light to fluctuate. The signal from detector 34 may also be indicative of this fluctuation, which can then be transmitted to processor 40 to be used to determine blood flow velocity. Advantageously, unlike the known method of measuring thermal conductivity to determine blood flow velocity, determining blood flow velocity in this manner does not require the additional steps of calibration and the measurement of water concentration in the skin. Zhao et al. (“Phase-Resolved Optical Coherence Tomography and Optical Doppler Tomography for Imaging Blood Flow in Human Skin with Fast Scanning Speed and High Velocity Sensitivity”, Opt. Lett., 25(2), pp 114-116 (2000)) have demonstrated the use of Doppler tomography to directly determine the blood flow rate.

As an alternative to an OCT device, an NIR diffuse reflectance device and detector could be used to measure the scattering coefficient which is dependent on refractive index. Advantageously the NIR diffuse reflectance device generates a signal indicative of the scattering coefficient at different wavelengths thereby providing more information.

The MHC method of determining blood glucose concentration requires determination of the total body heat, the skin surface temperature, the ambient temperature, the blood velocity and the concentration of haemoglobin and oxy-haemoglobin. As already disclosed the TES detector 16 can generate a signal indicative of the total body heat and indicative of the concentration of haemoglobin and oxy-haemoglobin and indicative of the ambient temperature. The OCT detector 34 can generate a signal indicative of the blood flow velocity and the system includes a thermistor 20 for measuring the skin surface temperature of the finger 1. The signals from the detectors 16 and 34 and the thermistor 25 are processed by processor 40 to determine the blood glucose concentration according to the known MHC method.

A separate thermistor for measuring the ambient temperature directly may be included in the system.

Because TES gives a direct glucose measurement and the MHC method and OCT methods give an indirect measurement, the factors influencing the blood glucose concentration measurements are different. Therefore the independent measurements can be compared to improve accuracy and combined to provide an average for the blood glucose concentration.

Referring to FIG. 2, the system elements corresponding to those in FIG. 1 are numbered in accordance with FIG. 1. The system comprises a pulsed superluminescent diode 51 and a photo-acoustic sensor 50. Pulsed light at a wavelength chosen to interact with the analyte e.g. glucose, is fired at the sample, finger 1. The light is absorbed by the analyte thereby generating microscopic local heating which results in a rapid rise in temperature. The temperature rise generates an ultrasound pressure wave 55, which is detected by photo-acoustic sensor 50 (e.g. a piezolelectric transducer made of lead metaniobate, lead zirconate titanate or polyvinylidene fluoride) on the surface of the skin. The magnitude of the pressure is proportional to the thermal expansion coefficient of the skin which is glucose dependent. The electric signal 52 generated by the sensor 50 is indicative of the thermal expansion coefficient of the skin of the subject and is transmitted to processor 40 which determines the blood glucose concentration therefrom. WO 2004/042382 discloses a method and apparatus for non-invasive measurement of living body characteristics by photoacoustics.

The signal indicative of the scattering coefficient generated by detector 34 may be used to isolate the thermo-elastic skin properties in the signal 52 generated by sensor 50 from scattering effects when the processor is determining the blood glucose concentration therefrom thereby increasing the accuracy of the blood glucose concentration value obtained.

Other spectroscopic devices suitable for use in the invention may include:

a raman spectroscopy device which generates a signal indicative of the concentration of haemoglobin and oxygenated haemoglobin in addition to glucose;
a fluorescent spectroscopy device which generates a signal indicative of glucose concentration;
a direct absorption spectrometer comprising an irradiator for irradiating a portion of the live subject with a measuring beam and a detector for collecting measuring beam radiation transmitted by said live subject and generating a signal indicative of the absorption of glucose in the portion of the subject. If the irradiator has multiple wavelengths, a signal also indicative of the concentration of haemoglobin and oxygenated haemoglobin can be generated.

Although a finger is shown in FIGS. 1 and 2 is should be understood that the system of the invention can be used with other body parts.

Although one common processor 40 has been illustrated the signals from each detector may be transmitted to separate processors before being at least partially combined.

It should be noted that the above-mentioned embodiments illustrate rather than limit the invention, and that those skilled in the art will be capable of designing many alternative embodiments without departing from the scope of the invention as defined by the appended claims. In the claims, any reference signs placed in parentheses shall not be construed as limiting the claims. The word “comprising” and “comprises”, and the like, does not exclude the presence of elements or steps other than those listed in any claim or the specification as a whole. The singular reference of an element does not exclude the plural reference of such elements and vice-versa. The invention may be implemented by means of hardware comprising several distinct elements, and by means of a suitably programmed computer. In a device claim enumerating several means, several of these means may be embodied by one and the same item of hardware. The mere fact that certain measures are recited in mutually different dependent claims does not indicate that a combination of these measures cannot be used to advantage.

1. A system for the non-invasive measurement of blood glucose concentration in a live subject comprising:
a. means for determining the body heat of the subject,
b. means for determining the concentration of haemoglobin and oxygenated haemoglobin in the blood of said live subject, and
c. means for determining blood flow velocity in respect of said live subject and means for determining blood glucose concentration in said live subject as a function of said body heat, said haemoglobin and oxygenated haemoglobin concentrations and said blood flow velocity; and
a plurality of spectroscopic devices each generating a signal indicative of blood glucose concentration, means for determining the blood glucose concentration from the signal indicative of blood glucose concentration and wherein at least one of the spectroscopic devices generates a signal additionally indicative of one or more of:
d. concentration of haemoglobin and oxygenated haemoglobin in the blood of the live subject;
e. the body heat of the live subject;
f. ambient temperature;
g. blood flow velocity in respect of said live subject,
wherein the signal indicative of one or more of d. to g. are transmitted to at least one of means a. to c. and used to determine the blood glucose concentration.

2. A system according to claim 1 wherein one of the spectroscopic devices comprises
x) a detector for detecting the thermal emission spectrum emitted by said live subject and generating a signal indicative of the absorption of glucose.

3. A system according to claim 1 wherein one of the spectroscopic devices comprises y) an irradiator for irradiating a portion of the live subject with a measuring beam and a detector for collecting measuring beam radiation scattered by said live subject and generating a signal indicative of the scattering coefficient of the portion of the subject.

4. A system according to claim 3, wherein the measuring beam is in the near infrared spectrum and/or has multiple wavelengths.

5. A system according to claim 1, wherein one of the spectroscopic devices comprises z) a source for pulsed irradiation of a portion of the live subject and a detector for detecting an acoustic pressure wave caused by the pulsed irradiation and generating a signal indicative of the thermo-elastic skin properties.

6. A system according to claim 3, wherein the signal indicative of the scattering coefficient is used to isolate the thermo-elastic skin properties in the signal indicative of the thermo-elastic skin properties from scattering effects.

7. A system according to claim 2, wherein the spectroscopy device comprises interference filtering means for spatially separating said thermal emission spectrum to create a plurality of spectral patterns and measuring in respect of each of a plurality of said spectral patterns a spectral intensity at a first, reference set of wavelengths, and a second set of wavelengths dependent on glucose or other analyte, and the concentration of glucose or other analyte is determined therefrom.

8. A system according to claim 7, wherein the interference filtering means comprises a spatial light modulator.

9. A system according to claim 8, wherein the interference filtering means comprises a multivariate optical element.

10. A system according to claim 7, wherein the signal generated by the detector of x) is also indicative of the concentration of haemoglobin and oxygenated haemoglobin in the blood of the live subject.

11. A system according to claim 2, wherein the signal generated by the detector of x) is also indicative of the body heat of the live subject.

12. A system according to claim 2, wherein the signal generated by the detector of x) is also indicative of the ambient temperature.

13. A system according to claim 3, wherein the irradiator and detector of y) are comprised in an optical coherence tomography device or optical Doppler tomography device.

14. A system according to claim 13, wherein the signal generated by the detector of y) is indicative of the blood flow velocity in respect of said live subject.

15. A method of determining blood glucose concentration in a live subject non-invasively comprising the steps of:
m. determining the body heat of the subject,
n. determining the concentration of haemoglobin and oxygenated haemoglobin in the blood of said live subject, and
o. determining blood flow velocity in respect of said live subject and means for determining blood glucose concentration in said live subject as a function of said body heat, said haemoglobin and oxygenated haemoglobin concentrations and said blood flow velocity; and
generating a signal indicative of blood glucose concentration from a plurality of spectroscopic devices and determining the blood glucose concentration therefrom, at least one signal being additionally indicative of one or more of:
p. concentration of haemoglobin and oxygenated haemoglobin in the blood of the live subject;
q. the body heat of the live subject;
r. ambient temperature;
s. blood flow velocity in respect of said live subject,
and using the signal(s) indicative of one or more of p. to s. in at least one of steps m to o.
